(19) United States
(12) Patent Application Publication
(10) Pub. No.: US 2012/0116186 A1
(43) Pub. Date: May 10, 2012

(54) METHOD AND APPARATUS FOR EVALUATION OF A SUBJECT'S EMOTIONAL, PHYSIOLOGICAL AND/OR PHYSICAL STATE WITH THE SUBJECT'S PHYSIOLOGICAL AND/OR ACOUSTIC DATA

(75) Inventors: Rahul Shrivastav, Gainesville, FL (US); Jenshan Lin, Gainesville, FL (US); Karl R. Zawoy, High Springs, FL (US); Sona Patel, Homer, IL (US)

(73) Assignee: UNIVERSITY OF FLORIDA RESEARCH FOUNDATION, INC., GAINESVILLE, FL (US)

(21) Appl. No.: 13/384,329

(22) PCT Filed: Jul. 20, 2010
(86) PCT No.: PCT/US2010/042603

§ 371 Date: Jan. 16, 2012

Related U.S. Application Data
(60) Provisional application No. 61/226,942, filed on Jul. 20, 2009.

(51) Int. Cl. A61B 5/00

(52) U.S. Cl. 600/301

(57) ABSTRACT

Embodiments of the subject invention relate to a method and apparatus for remote evaluation of a subject's emotive and/or physiological state. Embodiments can utilize a device that can be used to determine the emotional and/or physiological state of a subject through the measurement and analysis of vital signs and/or speech. A specific embodiment relates to a device capable of remotely acquiring a subject's physiological and/or acoustic data, and then correlating and analyzing the data to provide an assessment of a subject's emotional and/or physiological state. In a further specific embodiment, the device can acquire such data, correlate and analyze the data, and provide the assessment of the subject's emotional state and/or physiological in real time.

### CROSS-REFERENCE TO RELATED APPLICATION

The present application claims the benefit of U.S. Provisional Application Ser. No. 61/226,942, filed Jul. 20, 2009, which is hereby incorporated by reference herein in its entirety, including any figures, tables, or drawings.

### BACKGROUND OF INVENTION

There are many circumstances in which it is desirable to ascertain a person's emotional and/or physiological state. Typically, to make such a determination, a health care professional either interacts with the subject or the subject is hooked up to monitoring hardware, such as a lie detector device, in order to monitor the subject's physiological state, and further, derive conclusions about their emotional and/or physiological state. However, such conclusions about the subject's emotional and/or physiological state made by a health care professional can be subjective, as different health care professionals may reach different conclusions, and also, the rapport between the subject and the health care professional can influence the outcome. Further, hooking the subject up to monitoring hardware can be inconvenient and often impractical.

### BRIEF SUMMARY

Embodiments of the subject invention relate to a method and apparatus for evaluation of a subject's emotional and/or physiological state. Specific embodiments involve remote or partially remote, evaluation of a subject's emotional and/or physiological state. Embodiments can utilize a device that can be used to determine the emotional and/or physiological state of a subject through the measurement and analysis of the subject's physiological and/or acoustic data. A specific embodiment relates to a device capable of remotely acquiring a subject's physiological and/or acoustic data, and then correlating and analyzing the data to provide an assessment of a subject's emotional and/or physiological state. Such physiological data measured in accordance with embodiments of the invention can include any or all of the following: heartbeat, respiration, temperature, and galvanic skin response. Such acoustic data can include speech and/or non-verbal sounds. In a further specific embodiment, the device can acquire, correlate and analyze such data, and provide assessment of the subject's emotional and/or physiological state in real time.

### BRIEF DESCRIPTION OF DRAWINGS

FIG. 1 shows a schematic representation of an embodiment in accordance with the subject invention.

FIG. 2 shows acoustic measurements of pnorMIN and pnorMAX from the f0 contour.

FIG. 3 shows acoustic measurements of gtrend from the f0 contour.

FIG. 4 shows acoustic measurements of normnpks from the f0 contour.

FIG. 5 shows acoustic measurements of mpkrise and mpkfall from the f0 contour.

FIG. 6 shows acoustic measurements of iNmin and iNmax from the f0 contour.

FIG. 7 shows acoustic measurements of attack and dutycyc from the f0 contour.

FIG. 8 shows acoustic measurements of srtrend from the f0 contour.

FIG. 9 shows acoustic measurements of m_LTAS from the f0 contour.

FIG. 10 shows R-squared and stress measures as a function of the number of dimensions included in the MDS solution for 11 emotions.

FIG. 11 shows eleven emotions in a 2D stimulus space according to the perceptual MDS model.

FIG. 12 shows various characteristics related to emotion perception in accordance with embodiments of the subject invention.

FIG. 13 shows an emotion categorization scheme in accordance with an embodiment of the subject invention.

### DETAILED DISCLOSURE

Embodiments of the subject invention relate to a method and apparatus for evaluation of a subject's emotional and/or physiological state. Specific embodiments involve remote or partially remote, evaluation of a subject's emotional and/or physiological state. Embodiments can utilize a device that can be used to determine the emotional and/or physiological state of a subject through the measurement and analysis of the subject's physiological and/or acoustic data. A specific embodiment relates to a device capable of remotely acquiring a subject's physiological and/or acoustic data, and then correlating and analyzing the data to provide an assessment of a subject's emotional and/or physiological state. In a further specific embodiment, the device can acquire, correlate and analyze such data, and provide assessment of the subject's emotional state in real time.

Physiological data measured in accordance with embodiments of the invention can include any or all of the following: heartbeat, respiration, temperature, and galvanic skin response. Other vital signs known in the art can also be measured. As an example, galvanic skin response can be measured on a cell phone such as a flip-phone by placing two sets of electrodes on the surface of the phone. One set of electrodes can be located at the speaker and/or microphone area of the phone, and the other set of electrodes can be located on the outer surface of the phone where they can contact the subject's hand. In this way, when the subject holds the phone, the galvanic skin response can be measured. The measured galvanic skin response can then be used to measure stress, in a manner similar to a conventional lie detector test.

Acoustic data measured in accordance with embodiments of the invention can include, for example, patterns of speech, as well as patterns of non-verbal sounds such as bodily sounds from respiration, bodily sounds from digestion, breathing, and sounds unique to animals such as barking and chirping.

Embodiments can also measure physioacoustic (PA) data, which can be described as the simultaneous acquisition and measurement of physiological and acoustic data, including vital signs, voice, or other sounds derived from human or animal subjects. Physioacoustic data acquisition can directly correlate a subject's physiological response to sounds emanating from the subject.

Embodiments can also remotely measure physioacoustic (RPA) data, such that a subject's physioacoustic data is measured by way of a non-contact, or remote, measurement device.

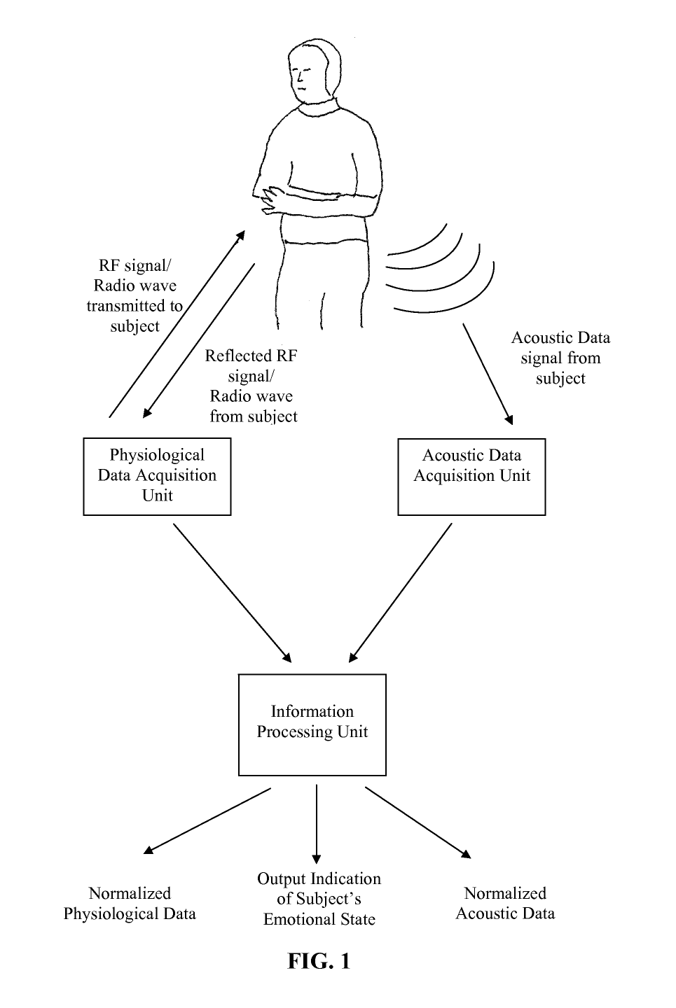

A remote physioacoustic device or system in accordance with an embodiment of the invention, such as the embodiment shown in FIG. 1, can incorporate a physiological data acquisition unit, an acoustic data acquisition unit, and an information processing unit. The system shown in FIG. 1 is an illustrative embodiment of the invention. Other embodiments of such a system may include more, fewer, or different components. Or the components shown may be differently arranged.

In specific embodiments, the physiological data acquisition unit can incorporate a method and apparatus of sensing or remote sensing of physiological data as taught in U.S. Publication No. U.S. 2008/0238757, which is incorporated by reference herein in its entirety, including any figures, tables, or drawings. In an embodiment, the physiological data acquisition unit can remotely detect, for example, a subject's cardiopulmonary or respiratory activity, by transmitting a double-sideband signal, such as a Ka-band electromagnetic wave with two frequency components, to the subject, and upon receiving the reflected electromagnetic wave, detect small motions emanating from the subject. Small motions that can be detected by the physiological data acquisition unit can include, for example, heartbeat-induced and/or respiration-induced changes in the chest wall of the subject.

In further specific embodiments, the physiological data acquisition unit can incorporate a method and apparatus of remote measurement of frequency and amplitude of mechanical vibration as taught in U.S. Publication No. U.S. 2008/0300805, which is incorporated by reference herein in its entirety, including any figures, tables, or drawings. In an embodiment, the physiological data acquisition unit can sense, for example, a subject's cardiopulmonary activity, by using a non-linear phase modulation method, to determine amplitude of the subject's periodic movement. Specifically, a physiological data acquisition unit in one embodiment transmits an RF signal towards the subject, receives the reflected RF signal from the subject, identifies the different orders of harmonics caused by a non-linear effect in the reflected RF signal, and determines the amplitude of the periodic movement of the subject from the identified different orders of harmonics. Alternatively, a physiological data acquisition unit in another embodiment first transmits and receives the reflected RF signal from the subject. Next, the unit down-converts the received RF signal to a baseband signal, from which a harmonic having an order n and an additional harmonic having an order n+2 are determined, wherein n is an integer. Then, a model is determined wherein the model uses the ratio of the n+2 order harmonic and the n order harmonic as a function of movement amplitude, and a measured ratio is calculated from a ratio of the n+2 order harmonic of the baseband signal and the one harmonic of the baseband signal. Last, the amplitude of the subject's periodic movement is determined by comparing the measured ratio to the model and selecting the amplitude corresponding to the measured ratio.

In still further specific embodiments, the physiological data acquisition unit can incorporate a method and apparatus of using remote Doppler radar sensing for monitoring mechanical vibration, as taught in WO Publication No. 2009/009690, which is incorporated by reference herein in its entirety, including any figures, tables, or drawings. In an embodiment, the physiological data acquisition unit can sense, for example, a subject's cardiopulmonary activity and respiration, by simultaneously transmitting electromagnetic waves, such as radio frequency (RF) waves, of at least two wavelengths, receiving the reflected electromagnetic waves, and subsequently extracting the subject's vibrational information from the reflected electromagnetic waves.

In yet further specific embodiments, the physiological data acquisition unit can incorporate a method and apparatus of remote vital sign detection, as taught in WO Publication No. 2009/076298, which is incorporated by reference herein in its entirety, including any figures, tables, or drawings. In an embodiment, the physiological data acquisition unit can recover detected signals from vibrating objects. Here, the physiological data acquisition unit transmits a signal to a subject and then receives a reflected signal from the subject. Then, the unit reconstructs a complex signal for the received reflected signal. Next, the unit applies a Fourier transform to the reconstructed signal, and obtains original vibration information for the subject by analyzing the angular information extracted from the reconstructed signal. By acquiring the original vibration information, the unit can obtain original body movement information, from which the unit obtains the subject's vital sign information.

The physiological data acquisition unit can include a non-contact detection radar, which detects, for example, a subject's vital signs. The non-contact detection radar transmits a radio wave toward a subject being monitored and receives a reflected radio wave from the subject. Information regarding the subject's physiological motions induced by heartbeat and respiration can be derived when information known about the transmitted radio wave is compared with information from the received reflected radio wave.

The acoustic data acquisition unit can collect acoustic data such as the speech and/or sounds produced by the subject being monitored. The acoustic data acquisition unit can incorporate a system and method of measurement of voice quality as taught in U.S. Publication No. 2004/0167774, which is incorporated by reference herein in its entirety, including any figures, tables, or drawings. In an embodiment, the acoustic data acquisition unit first processes the subject's voice using a model of the human auditory system, which accounts for the psychological perception of the listener. After processing the subject's voice through this model, the resulting signal is then analyzed using objective criteria to determine a measure of quality of voice such as breathiness, hoarseness, roughness, strain, or other voice qualities.

In specific embodiments, the acoustic data acquisition unit can incorporate a method and apparatus for speech analysis as taught in International Application No. PCT/US2010/038893, which is incorporated by reference herein in its entirety, including any figures, tables, or drawings. In an embodiment, the acoustic data acquisition unit can analyze speech, including the emotion associated with speech. From suprasegmental speech (SS) information the unit receives from the subject's speech, the unit can use, for example, unique dimensional attributes as determined in a multidimensional scaling (MDS) model, to determine perceptual characteristics used by listeners in discriminating emotions. In one embodiment, the unit can utilize four groups of acoustic features in speech including, but not limited to, duration measurements, fundamental frequency cues, vocal intensity cues, and voice quality.

In addition to these four acoustic features, other cues that have been previously investigated in the literature, such as speaking rate and f0, may be calculated using novel algorithms and used. A list of the acoustic cues taught in International Application No. PCT/US2010/038893, which is incorporated by reference herein in its entirety, including any figures, tables, or drawings is shown in Table 1.

TABLE 1
List of acoustic features analyzed.

| | | Abbre- |
|---|---|---|
| Feature Set | Acoustic Cues | viation |
| Fundamental | Global normalized f0 max | pnorMAX |
| frequency (f0) | Global normalized f0 min | pnorMIN |
| | Gross f0 trend | gtrend |
| | Normalized number of f0 contour peaks | normnpks |
| | Steepness of f0 contour peaks: Peak rise time | mpkrise |
| | Steepness of f0 contour peaks: Peak fall time | mpkfall |
| Intensity | Normalized Minimum | iNmin |
| | Normalized Maximum | iNmax |
| | Attack time of syllables in contour | attack |
| | Normalized attack time of syllables in contour | Nattack |
| | Normalized attack (by dutycyc) | normattack |
| | Duty cycle of syllables in contour | dutycyc |
| Duration | Speaking rate | srate |
| | Vowel to consonant ratio | VCR |
| | Pause Proportion | PP |
| | Speaking rate trend | strend |
| Voice quality | Breathiness-Cepstral peak prominence | MeanCPP |
| | Spectral tilt-alpha ratio of stressed vowel | aratio |
| | (summed) | |
| | Spectral tilt-mean alpha ratio of stressed vowel | maratio |
| | Spectral tilt-regression through the long-term | mLTAS |
| | averaged spectrum of stressed vowel | |
| | Spectral tilt-regression through the long-term | mLTAS2 |
| | averaged spectrum of unstressed vowel | |
| | Spectral tilt-mean alpha ratio of unstressed | maratio2 |
| | vowel | |
| | Spectral tilt-alpha ratio of unstressed vowel | aratio2 |
| | (summed) | |

Many of these acoustic parameters can be estimated by dividing the speech signal into small time segments or windows, and this process can be used to capture the dynamic changes in the acoustic parameters in the form of contours. It is often convenient to smooth the contours before extracting features from these contours. As a result, a preprocessing step may be performed prior to computing some acoustic features. Acoustic measures can also be computed manually.

### Dimension Models

An acoustic model of emotion perception in SS can be developed through a multidimensional scaling study and then performing a feature selection process to determine the acoustic features that correspond to each dimension MDS model. The significant predictors and their coefficients for one MDS model are summarized in regression equations shown in Table 2.

TABLE 2
Regression equations for multiple perceptual models using the training and test₁ sets.

| | | | Regression Equation |
|---|---|---|---|
| TRAINING | Overall | D1 | −0.002*aratio2 − 0.768*srate − 0.026*pnorMIN + 13.87 |
| | | D2 | −0.887*normattack + 0.132*normpnorMIN − 1.421 |
| | Spk1 | D1 | −0.001*aratio + 0.983*srate + 0.256*Nattack + 4.828*normnpks + 2.298 |
| | | D2 | −2.066*attack + 0.031*pnorMIN + 0.097*iNmax − 2.832 |
| | Spk2 | D1 | −2.025*VCR − 0.006*mpkfall − 0.071*pnorMIN + 6.943 |
| | | D2 | −0.662*normattack + 0.049*pnorMIN − 0.008*mpkrise − 0.369 |
| | Overall | D1 | −0.238*iNmax − 1.523*srate − 0.02*pnorMAX + 14.961*dutycyc + 4.83 |
| | | D2 | −1.584*srate + 0.013*mpkrise − 12.185*srtrend − 12.185 |
| | Spk1 | D1 | 0.265*iNmax − 7.097*dutycyc + 0.028*pnorMAX + 0.807*MeanCPP − 16.651 |
| | | D2 | 0.036*normpnorMIN + 7.477*PP − 524.541*m_ LTAS + 0.159*maratio2 − 2.061 |
| | Spk2 | D1 | 0.249*iNmax + 14.257*dutycyc − 0.011*pnorMAX − 0.071*pnorMIN − 6.687 |
| | | D2 | −0.464*iNmax + 0.014*MeanCPP + 7.06*normnpks + 7.594*srtrend − |
| | | | 2.614*srate − 14.805 |
| TEST₁ | Sent1 | D1 | 0.178*iNmin − 1.677*srate + 0.025*pnorMAX − 0.028*pnorMIN + 1.446 |
| | | D2 | −0.003*aratio − 3.289*VCR − 0.007*mpkfall + 0.008*pnorMAX + 22.475 |
| | Sent2 | D1 | 4.802*srtrend − 0.044*pnorMIN − 0.013*pnorMAX + 4.721 |
| | | D2 | −7.038*srtrend + 0.017*pnorMAX − 1.47*srate + 0.201*normattack + 2.542 |
| | Spk1, | D1 | −0.336*maratio + 0.008*mpkrise + 0.206*iNmin − 0.122*maratio2 − 10.306 |
| | Sent1 | D2 | −0.006*mpkrise − 15.768*dutycyc − 0.879*MeanCPP − 0.013*pnorMIN + 21.423 |
| | Spk1, | D1 | −6.68*normnpks + 0.221*iNmax − 0.002*aratio + 270.486*m_LTAS + 10.171 |
| | Sent2 | D2 | −28.454*gtrend + 0.504*maratio2 − 0.038*pnorMIN − 0.193*iNmin − |
| | | | 736.463*mLTAS2 − 0.992*MeanCPP + 24.581 |
| | Spk2, | D1 | −0.034*pnorMAX − 8.336*srtrend + 0.002*aratio − 2.086*VCR − 5.438 |
| | Sent1 | D2 | −0.334*maratio − 0.184*iNmin + 0.925*srate + 0.008*pnorMAX − 4.197 |
| | Spk2, | D1 | −0.304*maratio2 − 591.928*m _LTAS2 + 0.139*normpnorMIN − 11.395 |
| | Sent2 | D2 | 298.412*m LTAS + 7.784*VCR − 0.007*mpkfall + 156.11*PP + |
| | | | 0.091*pnorMIN − 0.002*aratio − 1.884 |

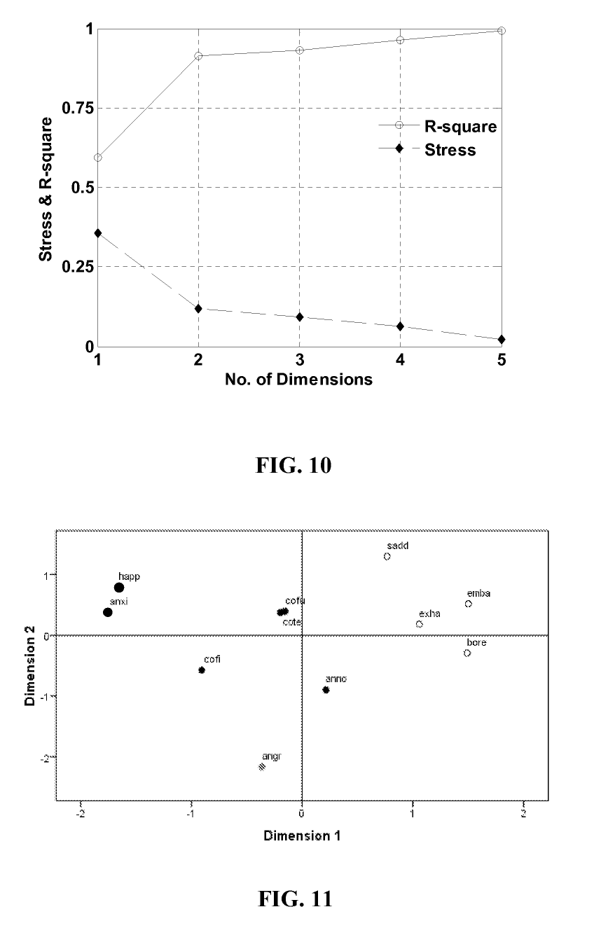

These equations may form the acoustic model and be used to describe each speech sample in a 2D acoustic space. For example, the acoustic model that describes the “Overall” training set model can include the parameters aratio2, srate, and pnorMIN for Dimension 1 (parameter abbreviations are outlined in Table 1). These cues can be predicted to correspond to Dimension 1 because this dimension separates emotions according to energy or “activation”, whereas Dimension 2 was described by normattack (normalized attack time of the intensity contour) and normpnorMIN (normalized minimum pitch, normalized by speaking rate) since Dimension 2 seems to perceptually separate angry from the rest of emotions by a staccato-like prosody. Alternatively, Dimension 1 may be described by iNmax (normalized intensity maximum), pnorMAX (normalized pitch maximum), and dutycyc (duty cycle of the intensity contour). Dimension 2 may be predicted by srate, mpkrise (mean f0 peak rise time) and srtrend (speaking rate trend). In other embodiments, a three or more dimension acoustic space can be formed having at least one SS or other acoustic cues corresponding to each dimension. An emotion state of a subject can be described using at least one magnitude along a corresponding at least one of the dimensions within the acoustic space. FIG. 10 shows R-squared and stress measures as a function of the number of dimensions included in the MDS solution for 11 emotions. FIG. 11 shows eleven emotions in a 2D stimulus space according to the perceptual MDS model.

The following sections describe the methods for calculating acoustic cues, such as fundamental frequency, intensity, duration, and voice quality.

1. Fundamental Frequency (f0)

A number of static and dynamic parameters based on the fundamental frequency can be calculated in order to provide an indicator of the subject's emotional and/or physiological state. To obtain these measurements, the f0 contour can be computed using a variety of algorithms such as autocorrelation or SWIPE’ (Camacho, 2007, incorporated by reference herein in its entirety, including any figures, tables, or drawings). The SWIPE’ algorithm is preferred in this application since it has been shown to perform significantly better than other algorithms for normal speech (Camacho, 2007). However, any of the several methods available to compute fundamental frequency may be used. Alternately, algorithms to compute pitch may be used instead. Pitch is defined as the perceptual correlate of fundamental frequency.

Once the f0 contours are computed, they can be smoothed and corrected prior to making any measurements. The pitch minimum and maximum may then be computed from final pitch contours. To normalize the maxima and minima, these measures can be computed as the absolute maximum minus the mean (referred to as “pnorMAX” for normalized pitch maximum) and the mean minus the absolute minimum (referred to as “pnorMIN” for normalized pitch minimum). This is shown in FIG. 2.

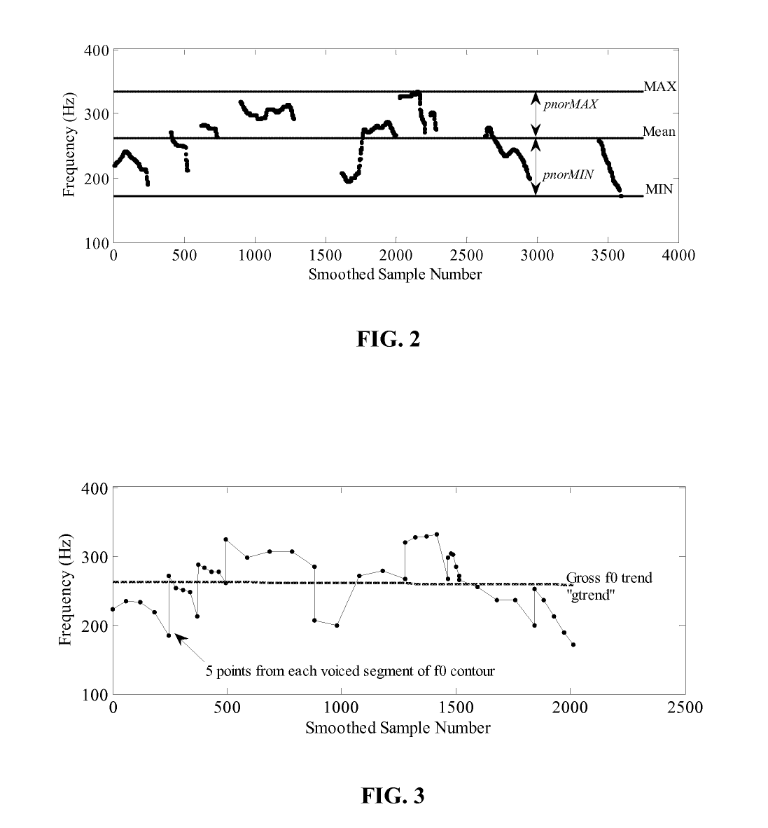

A number of dynamic measurements may also be made using the contours. Dynamic information may be more informative than static information in some situations. These include measures such as the gross trend (“gtrend”), contour shape, number of peaks, etc. Gross trend may be computed by fitting a linear regression line to the f0 contour and computing the slope of this line, as shown in FIG. 3.

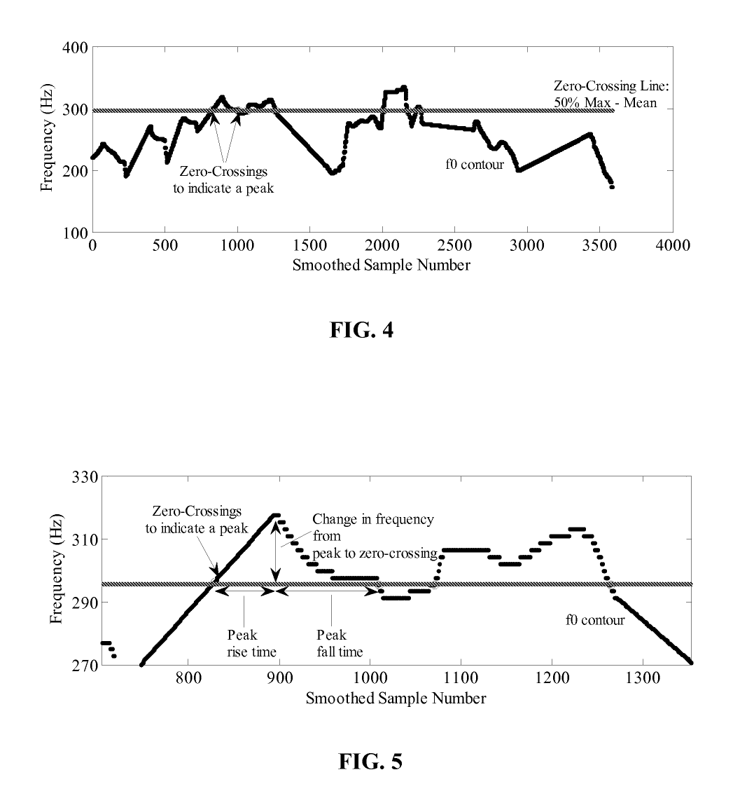

The contour shape may be quantified by the number of peaks in the f0 contour, which may be measured using any available peak-picking algorithms. For example, zero-crossings can indicate as peak, as shown in FIG. 4. The normalized number of f0 peaks (“normnpks”) parameter can then be computed as the number of peaks in the f0 contour divided by the number of syllables within the sentence. Another method used to assess the f0 contour shape is to measure the steepness of f0 peaks. This can be calculated as the mean rising slope and mean falling slope of the peak. The rising slope (“mpkrise”) can be computed as the difference between the maximum peak frequency and the zero crossing frequency, divided by the difference between the zero-crossing time prior to the peak and the peak time at which the peak occurred (i.e., the time period of the peak frequency or the “peak time”). Similarly, the falling slope (“mpkfall”) can be computed as the difference between the maximum peak frequency and the zero crossing frequency, divided by the difference between the peak time and the zero-crossing time following the peak. The computation of these two cues is shown in FIG. 5. These parameters can be further normalized by the speaking rate, since fast speech rates can result in steeper peaks. The formulas for these parameters are as follows:

peakᵣᵢₛₑ=[(f_{peak max}−t_{zero-crossing})/(t_{peak max}−t_{zero-crossing})]/speaking rate (1)

peak_{fall}=[(f_{peak max}−t_{zero-crossing)})/(t_{zero-crossing}−t_{peak max})]/speaking rate (2)

The peakᵣᵢₛₑ and peak_{fall} can be computed for all peaks and averaged to form the final parameters mpkrise and mpkfall.

In various embodiments, cues that can be investigated include fundamental frequency as measured using SWIPE’, the normnpks, and the two measures of steepness of the f0 contour peaks (mpkrise and mpkfall). These cues may provide better classification of emotions in SS, since they attempt to capture the temporal changes in f0 from an improved estimation of f0.

### 2. Intensity

Intensity is essentially a measure of the energy in the speech signal. In specific embodiments, the intensity of each speech sample can be computed for 20 ms windows with a 50% overlap. In each window, the root mean squared (RMS) amplitude can be determined and then converted to decibels (dB) using the following formula:

Intensity(dB)=20*log₁₀ [mean(amp²)]^{1/2} (3)

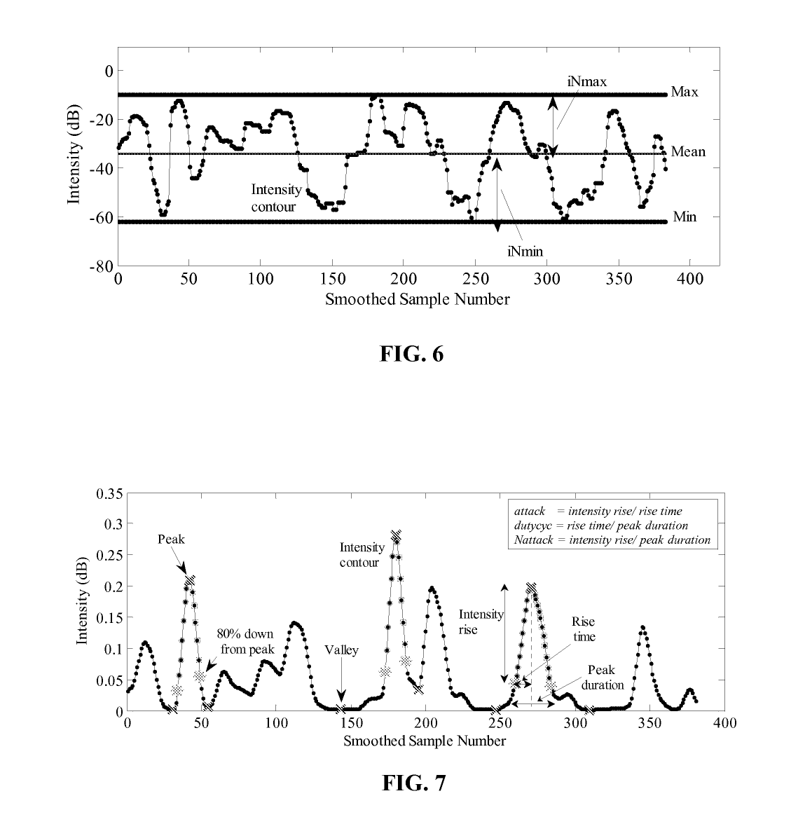

The parameter amp refers to the amplitude of each sample within a window. This formula can be used to compute the intensity contour of each signal. The global minimum and maximum can be extracted from the smoothed RMS energy contour. The intensity minimum and maximum can be normalized for each sentence by computing the absolute maximum minus the mean (referred to as “iNmax” for normalized intensity maximum) and the mean minus the absolute minimum (referred to as “iNmin” for normalized intensity minimum), as shown in FIG. 6.

In addition, the duty cycle and attack of the intensity contour can be computed as an average across measurements from the three highest peaks. The duty cycle (“dutycyc”) can be computed by dividing the rise time of the peak by the total duration of the peak. The attack (“attack”) can be computed as the intensity difference for the rise time of the peak divided by the rise time of the peak. The normalized attack (“Nattack”) can be computed by dividing the attack by the total duration of the peak, since peaks of shorter duration would have faster rise times, and another normalization can be performed by dividing the attack by the duty cycle (“normattack”). This can be performed to normalize the attack to the rise time as affected by the speaking rate and peak duration. The computations of attack and dutycyc are shown in FIG. 7.

### 3. Duration

Speaking rate (i.e., rate of articulation or tempo) can be used as a measure of duration and calculated as the number of syllables per second. An estimation of syllable boundary can be made using the intensity contour. This can be effective with speech in the English language, as all English syllables form peaks in the intensity contour. The peaks are areas of higher energy, which typically result from vowels, and since all syllables contain vowels, they can be represented by peaks in the intensity contour. The rate of speech can then be calculated as the number of peaks in the intensity contour. Therefore, the speaking rate (“srate”) is the number of peaks in the intensity contour divided by the total speech sample duration.

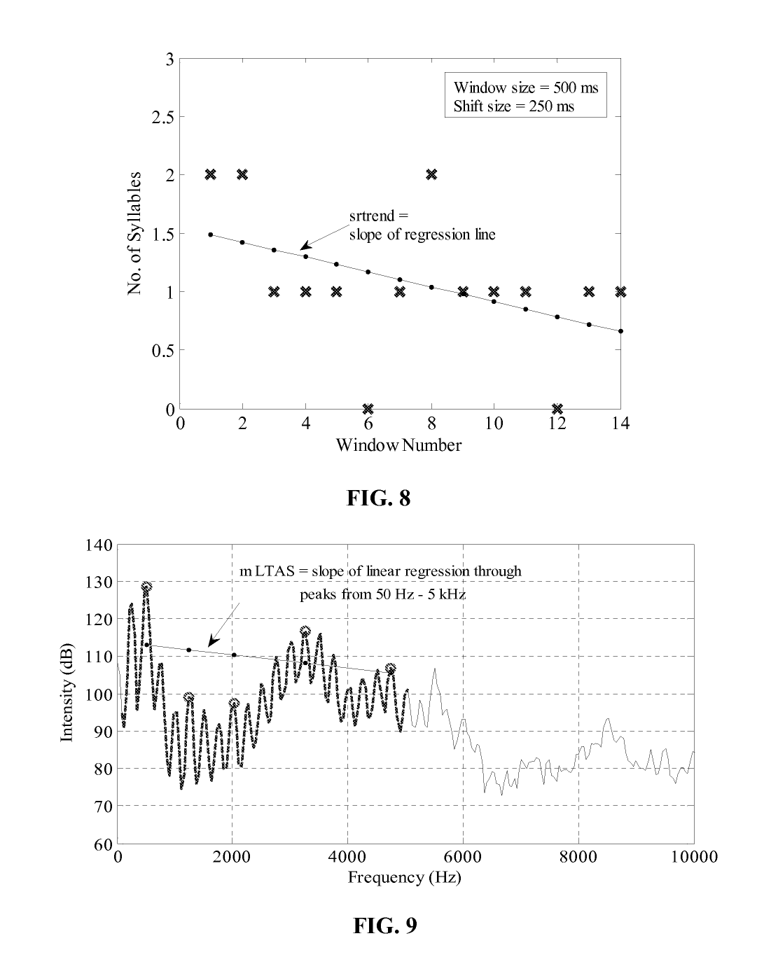

In addition, the number of peaks in a certain window can be calculated across the signal to form a “speaking rate contour” or an estimate of the change in speaking rate over time. The slope of the best fit linear regression equation through these points can then be used as an estimate of the change in speaking rate over time or the speaking rate trend (“srtrend”), the calculation of which is shown in FIG. 8.

In addition, the vowel-to-consonant ratio (“VCR”) can be computed as the ratio of total vowel duration to the total consonant duration within each sample. The pause proportion (the total pause duration within a sentence relative to the total sentence duration or “PP”) can also be measured and is defined as non-speech silences longer than 50 ms. Since silences prior to stops may be considered speech-related silences, these are not considered pauses unless the silence segment was extremely long (i.e., greater than 100 ms).

4. Voice quality

Many experiments suggest that anger can be described by a tense or harsh voice (Scherer, 1986; Burkhardt & Sendlmeier, 2000; Gobl and Chasaide, 2003, incorporated by reference herein in their entirety, including any figures, tables, or drawings). Therefore, parameters used to quantify high vocal tension or low vocal tension (related to breathiness) may be useful in describing emotions. One such parameter is the spectral slope. Spectral slope may be useful as an approximation of strain or tension (Schroder, 2003, p. 109, incorporated by reference herein in its entirety, including any figures, tables, or drawings), since the spectral slope of tense voices is shallower than that for relaxed voices. Embodiments can measure the spectral slope using, for example, one of two methods. In the first method, the alpha ratio can be computed (“aratio” and “aratio2”). This is a measure of the relative amount of low frequency energy to high frequency energy within a vowel. To calculate the alpha ratio of a vowel, the long term averaged spectrum (LTAS) of the vowel can be computed first. Then, the total RMS power within the 1 kHz to 5 kHz band can be subtracted from the total RMS power in the 50 Hz to 1 kHz band. An alternate method for computing alpha ratio computes the mean RMS power within the 1 kHz to 5 kHz band and subtracts it from the mean RMS power in the 50 Hz to 1 kHz band (“maratio” and “maratio2”). This second method for measuring spectral slope determines the slope of the line that fits the spectral peaks in the LTAS of the vowels (“m_LTAS” and “m_LTAS2”). A peak-picking algorithm can then be used to determine the peaks in the LTAS. Linear regression may then be performed using these peak points and the slope of the linear regression line may be used as the second measure of the spectral slope as shown in FIG. 9. The cepstral peak prominence (CPP) may be computed as a measure of breathiness as described by Hillenbrand and Houde (1996), which is incorporated by reference herein in its entirety, including any figures, tables, or drawings.

### Model Classification Procedures

Once the various acoustic cues have been computed, these can be used to classify a speech utterance into a particular emotion category. The acoustic cues for each dimension are used to locate each sample on an MDS space. This location is then used to classify that sample into one of four emotion categories using an appropriate classification algorithm such as the k-means algorithm.

In specific embodiments, the acoustic data acquisition unit can acquire speech and/or other acoustic signals by using an appropriate transducer (microphone), connected to a signal acquisition system (e.g., analog-to-digital converted, storage device). A suitable impedance matching device, such as a preamplifier, can be added. Once recorded, the speech is analyzed to derive specific parameters, and the analysis routine can involve several steps. First, several pre-processing steps may be applied to make the acoustic data signals suitable for further analyses. For example, simple filters or more complex algorithms may be used for noise reduction. For derivation of specific parameters, the signal may need to be passed through an “auditory front-end.” This auditory front-end can simulate one or more of the processes involved in the transduction of acoustic signals in human auditory pathways in order to provide a closer approximation to how sound may be processed by humans. These pre-processing steps may also involve specific methods for segmenting the input signal (such as based on fixed-time units, or based on more complex criteria such as syllable-boundary detection or word detection). Analysis of the acoustic signals involves estimation of specific parameters or measures from the signal. These parameters describe specific characteristics of the input signal, and are often derived from short segments of the input signal. Some parameters may be derived from short fixed-interval segments (“windows”) while others may be derived from more complex segmentation criteria (phrase-level, word-level, syllable-level). The parameter of interest may be the average value across one or more segments, or patterns/degree of change in these values across multiple segments. The measures may be obtained from the acoustic waveform or the spectrum or some derivation of these representations. Measures may pertain to multiple aspects of the input signal, such its fundamental frequency, intensity and various spectral characteristics including formant frequencies, spectral shape, relative noise levels, and/or other characteristics.

The physiological data from the physiological data acquisition unit, and the acoustic data from the acoustic data acquisition unit can then be sent to the information processing unit. The information processing unit can collects this data and processes the data from both units in real time, or at a later time, and makes assessments based on the program designed for a specific application. The parameters derived from the signal analyses are then used for decision making in the information processing unit using one or more of a number of different algorithms. For example, decisions maybe based on a linear or non-linear combination of multiple parameters as derived from a regression function for a set of data. More complex classification or pattern-recognition approaches may also be used. These include, for example, artificial neural networks (ANN), hidden Markov models (HMM), and support vector machines (SVM). The results from the information processing unit can then be displayed on a screen or recorded in data storage media.

Combining information obtained from physiological and acoustic signals provides a powerful tool, especially for remote applications, because the two streams of information may be complementary or supplementary to each other. When the streams of information are complementary to each other, they provide more information than either alone. Alternatively, when the streams of information are supplementary to each other, they can increase the accuracy obtained by either stream of information alone. Depending upon the particular application, the information from the two sets of data may be combined in different ways.

In some embodiments of the invention, the acoustic signals may be used to derive information about the subject that is used to normalize or correct the physiological data. For example, heart rate or respiration rate may vary as a function of age and/or a change in emotional status. The acoustic signal may be used to estimate the subject's age or emotional status and this may then be used to normalize (or correct) the physiological data before making additional decisions. Alternatively, information gathered from physiological data may be used to normalize specific acoustic measures.

In other embodiments of the invention, the information from the physiological and acoustic data streams may be combined to increase the efficiency or accuracy of decisions. For example, in an application to monitor stress levels, physiological and acoustic data may be combined to determine the level of stress for a subject. In general, the combination of data may take one or more of the following forms:
1. Physiological and acoustic data, such as vital sign and speech data, serves as input to an information processing unit.
2. Data from one source is used to normalize the other.
3. The raw data (with or without normalization) is sent to a decision engine in the information processing unit. The decision engine may involve relatively simple decision trees, linear or non-linear regression equations, and/or more complex pattern recognition algorithms.

In an embodiment of the subject invention, an “assessment model” can be loaded into the information processing unit and run using the model run based on the physiological data, such as voice, heartbeat, and respiration data, and acoustic data received from the acquisition units. The information processing unit can also be programmed based on the type of emotional and/or physiological analysis of the subject that is desired. In one embodiment, empirical data derived from clinical trials, or other sources (based on an expanded set of “wired” physiological measurements) can be used in order to derive a reduced set based on acquired data such as voice, heartbeat, respiration and temperature (infrared). Alternatively, in another embodiment, empirical data derived from user feedback can be used in order to derive a reduced variable set based on this acquired data. In an embodiment, an assessment model used to analyze consumer emotions after purchasing of a product as illustrated in Westbrook, R. A. et. al., “The Dimensionality of Consumption Emotion Patterns and Consumer Satisfaction”, Journal of Consumer Research, Inc., Vol. 18, 1991, which is incorporated by reference herein in its entirety, including any figures, tables, or drawings, can be loaded into the information processing unit. This assessment model can use, for example, taxonomic and dimensional analyses to identify patterns of emotional and/or physiological response to certain experiences, such as product experiences. In another embodiment, an psychoanalytic assessment model can also be loaded into the information processing unit in order to rate the subjects emotional level. In an embodiment, a psychoanalytic assessment model similar to the model used in Benotsch, E. G., “Rapid Anxiety Assessment in Medical Patients: Evidence for the Validity of Verbal Anxiety Ratings”, Annals of Behavioral Medicine, 2000, pp. 199-203, which is incorporated by reference herein in its entirety, including any figures, tables, or drawings, may also be loaded into the information processing unit and subsequent analysis of the physiological and acoustic data from the acquisition units performed.

### Example 2

### Remote Screening for Post Traumatic Stress Disorder (PTSD)

A physioacoustic (PA) screening tool for PTSD may take the following form:

1. The subject to be tested is asked a series of questions, either in a live interview with a health care professional or in a remote interview, for example, over telephone or Voice IP.

2. The subject's various physiological and acoustic, for example, speech, signals are recorded and monitored, either offline or in real-time.

3. The speech signals may optionally be used to estimate the age and gender of the subject for example if not otherwise provided.

4. The subject's estimated age and gender, or provided age and gender are then used to identify the normative range of other speech parameters as well as various physiological data, such as heart rate or respiration.

5. The changes in various physiological and speech data in response to specific questions can then be tracked.

6. The physiological and speech data are then sent to an information processing unit that is able to process and combine these individual physiological and speech signals, compare it to the subject's age and gender (also, possibly other factors such as ethnicity), and issue a decision regarding the likelihood of PTSD in that subject. For example, it may be the case that subjects with PTSD tend to have a greater change in heart rate, respiration (mean or variability) or specific speech parameters from the baseline (even after accounting for age, gender, or ethnicity) in response to the same set of questions than is seen in subjects without PTSD. The relevant parameters are subject to empirical study, but may include data such as mean heart rate, short-term and long-term variability in heart rate, short-term and long-term variability in galvanic skin response, temperature, respiration, fundamental frequency of speech, intensity and/or power of speech, changes in voice quality, patterns of changes in fundamental frequency, intensity, syllabic duration in speech, as well as other data. The information processing unit will then issue a statistical probability stating the likelihood of PTSD in patients with similar behavior patterns.

### Example 3

### Real-Time Assessment of Effort

A real-time assessment of effort may be useful in several applications where optimal levels of effort is critical for job performance, such as for pilots or crane operators. The effort levels may be monitored in real-time using the collection and assessment of physioacoustic (PA) data. For instance, a suitable device for remote measurement of PA signals may be installed in the cockpit of a crane. When in operation, the system can monitor, for example continuously monitor, changes in heart-rate, respiration patterns and/or speech patterns of the crane operator. These physiological and speech signals can then be sent to an information processing unit that extracts relevant measures/features from each physiological signal train. For example, measures of interest may include the mean values of heart rate, respiration, vocal fundamental frequency, and speaking rate over select time frames. Other measures may include the short/long term variability in these signals or patterns of changes over time (such as a systematic rise and fall of a particular measure). The relevant information may be obtained through measurement of absolute change in these measures, or patterns of change across multiple parameters (e.g., simultaneous change in two or more parameters). All relevant information will be processed to issue a decision (likely based on statistical probability) regarding the level of effort being applied by an individual. If the effort level drops below a specific threshold value, an appropriate warning signal may be issued to alert the crane operator and/or others (e.g. supervisors).

An embodiment of a device in accordance with the subject invention can incorporate hardware and software that allow the device to be portable and/or integrated into a cell phone, laptop computer, or other portable electronic device. The remote physioacoustic RPA data acquisition technology can be implemented as a dedicated chip set, which can be programmed for, for example, numerous consumer, medical, and military applications. The device can also collect and send RPA data from one location to another location via, for example, a wireless signal. The device can also have a stealth mode where the device can operate while the subject is not aware that he or she is being evaluated.

An embodiment of the device can also be used to measure data that can be used to evaluate a subject's emotional and/or physiological state. For example, evaluation of the subject's emotional state can be used for the purpose of determining the probability that a subject exhibits certain behaviors, such as behaviors relating to post traumatic stress disorder (PTSD). The subject can be asked a series of questions, either by a health care practitioner or through a remote system accessed through, for example, the subject's cell phone or other communication device. As the subject answers the questions, RPA data can be collected, analyzed, and presented to the health care practitioner or remote data acquisition system, such as an embodiment of the subject invention. In this way, the practitioner can be provided with an assessment of the subject's state of mind based on the acquired RPA data, and can alter therapy and measure results in real-time, as the subject's therapy is altered. RPA data can also be collected from the patient numerous times a day to provide a more accurate assessment of the patent's emotional and/or physiological state over time.

In another embodiment, a device utilizing the techniques of the subject invention can also be used to enhance the effectiveness of existing lie detection systems, or act as a lie detection system without the use of cumbersome wires and electrodes. The device can be a portable lie detection system, and can be built into a portable electronic device, such as a cell phone. Vital sign data, such as heartbeat rhythm or breathing patterns, can be correlated to spoken sentences so as to provide the interviewer with additional physiological information about the subject.

Embodiments can also be applied to biometric devices. Such a device can be used to implement a non-contact method to verify the identity of a subject based on tightly correlated voice print and/or vital sign measurement data. A subject's spoken words can be correlated to, for example, heart beat rhythm and/or breathing patterns measured while the subject is speaking in order to provide a unique fool-proof biometric signature.

An embodiment can also be used to determine the emotional and/or physiological state of a witness during a trial at a distance. This can be accomplished without the witness knowing that he or she is being monitored. The remote physioacoustic device can be used to determine the emotional and/or physiological state of a speaker, again without the speaker knowing that he or she is being monitored, if desired.

Embodiments of the remote physioacoustic device can also be applied in a covert intelligence setting to determine the emotional and/or physiological state of a subject. Again, such a determination can be accomplished without the subject knowing that he or she is being monitored. In addition, the device can be integrated with a hidden microphone and small radio frequency antenna. Embodiments can take different shapes, such as the shape of a piece of jewelry to be worn by an agent. The device's output of the subject's emotional and/or physiological state can take the form of a simple signal such as a vibration on the user's belt, a text message sent to a cell phone, or an auditory response sent to a Bluetooth® headset or digital hearing aid.

Embodiments can also be used as a tool to assist a veterinarian in diagnosing the emotional or physiological state of animals, such as race horses, racing dogs, dolphins, and whales. The device can remotely correlate heartbeat, respiration, and/or breathing patterns with auditory signals from the animal, including the sound of breathing, barking, high pitched squeals, or other sounds. Results can then be used to determine the level of stress or fatigue and/or to measure the animal's response to intervention and treatment.

Embodiments can further be used in security applications where it is necessary to determine the quantity, age, gender, and/or relative health of people in a room or enclosed space. In addition, the device can be used to count the number of people based on their voice signatures and then determine vital signs and emotional and/or physiological states of the subjects. The device can be placed in the room and remotely activated and monitored.

Embodiments can also be used to continuously monitor comatose or severely handicapped patients in hospital or nursing home settings. Vital signs can be correlated to voice patterns or sounds by the patient or correlated to sounds of the patient's movement.

Embodiments can be used to monitor drug compliance by a patient or to diagnostic patient readings remotely by the physician. First, the patient can be called on a cell phone by a health care practitioner. Next, patients can be instructed to take their medication and stay on the phone. The patient's vital signs and auditory data can be acquired via the cell phone and correlated in real time and displayed on the health care practitioner's computer screen where they are calling from. The practitioner can then instruct the patient as to what to do next. If preferred, the acquired data can be correlated offline at a later time.

Embodiments of the invention can be also used to monitor the emotional and/or physiological state of crowds or fans from a remote location by pointing a dish microphone coupled with a radio frequency antenna at selected members in the crowd. Signals can be multiplexed to perform real-time remote physioacoustic analysis of a particular crowd member's emotional and/or physiological state.

The device can be integrated into appliances, such as smart appliances, to determine whether someone is in a room and if so, to ask them if they need something. An embodiment of the device can be integrated into a car to predict the emotional and/or physiological state of the driver. The device can be used to prevent road rage or to disable the car if a driver is out of control, experiencing a medical emergency such as cardiac arrest, or slurring words due to intoxication.

An embodiment can be integrated into a point-of-purchase display in a department store or other retail location. The device can detect the presence of a potential customer and assess whether the customer is, for example, relaxed, or in an emotional and/or physiological state to possibly make a purchase.

The subject remote physioacoustic technology can also be integrated into computers and portable devices to enhance the operation of a natural language interface or user interface. The technology can improve the collection and analysis of the spoken word by correlating a user's physioacoustic data with a user's interactions with the machine interface.

An embodiment of a remote physioacoustic device can also be used to correlate and quantify a patient's initial and follow-up response to cognitive therapy techniques in order to provide enhanced cognitive therapy techniques. Applications can include improving diagnosis of disorders using instruments such as The Burns Anxiety Inventory and Burns Depression Checklist [Reference David Bums, MD, The Feeling Good Handbook, 1984], which is incorporated by reference herein in its entirety, including any figures, tables, or drawings, to measure the emotional response to questions during the patient interview and after treatment.

An embodiment can use a remote physioacoustic device to perform early diagnosis of diseases such as Parkinson's Disease, Alzheimer's Disease, or other conditions where a subject's voice and vital signs are affected.

A remote physioacoustic device can be used to screen drivers for alcohol or drug abuse through the remote measurement of a patient's vital signs and voice patterns and comparison of the acquired vital signs and voice patterns to a control or pre-recorded sample taken at a previous time under normal conditions.

A remote physioacoustic device can be used in applications involving psychotherapy or neurolinguistic programming exercised where the therapist's voice is also recorded with the subject's voice and vital signs. The therapist's speech and related techniques can then be correlated to the patient's emotional and/or physiological response to determine the effect the therapist is having on the patient.

A remote physioacoustic device can be used to enhance the effectiveness of established techniques to determine the emotional and/or physiological state of the subject, for example: A new test of human emotion and/or physiological processing.

Using Dr. Paul Ekman's internationally-normed faces, the Comprehensive Affect Testing System (CATS) provides a well-validated, reliable computerized test of human emotion processing. The CATS provides clinical and research professionals with a tool to efficiently determine the subtle multidimensional deficits in emotion processing that can result from disease or injury. This ensemble of emotion tests enables clinical psychologists, neuropsychologists, neurologists, educators, speech therapists, and professionals in other related disciplines to assess dysfunctional processing of emotion expressed by the human face and voice. Thirteen subtests help differentiate specific areas of dysfunction that individual patients can exhibit relative to normal populations during emotion processing, as taught in http://www.psychologysoftware.com/CATS.htm, which is incorporated by reference herein in its entirety, including any figures, tables, or drawings.

An embodiment of the remote physioacoustic device can be integrated into home devices, such as bathroom fixtures or kitchen appliances and can monitor changes in a patient's health status remotely. The device may be a stand-alone unit or be integrated into a network. The device can be enabled to automatically run periodic tests on the patient and issue alerts or warnings to seek professional help if needed.

A remote physioacoustic device can produce signals that can be used to measure changes in a subject's effort during a particular listening task. These measured changes in effort can help guide the tuning of listening devices such as mobile phones or hearing aids so that listeners require minimal effort to achieve maximum performance.

A remote physioacoustic device can be used to monitor stress levels in people in critical tasks and to take remedial action as, and when, necessary, thereby minimizing the errors and accidents. As an example, the stress levels of workers such as crane operators, nuclear power plant workers, and airline pilots can be monitored during their regular work activity to ensure optimum attention levels. A warning signal may be provided if attention level drops below a critical level and alternative actions may be taken if the stress increases to a point that it may interfere with accurate performance.

A remote physioacoustic device can be integrated into a game console or computer to monitor the player's emotional and/or physiological status and feedback the emotional and/or physiological status to the game to dynamically alter the response. Such a device can enhance the human/machine interface.

A remote physioacoustic device can be used to monitor a pilot's vital sign condition. This would be especially useful for fighter jet pilots.

A remote physioacoustic device can be used in game shows or other contests, such as the JEOPARDY® TV show, to display contestants' heart rate and respiration rate variability in real time. The voice can be analyzed and displayed to show the level of correlation. The device can also be used to monitor poker players.

In an embodiment of the subject invention, a method of determining an emotional state of a subject is provided. In an embodiment, the method includes measuring one or more physiological characteristics of the subject and/or measuring one or more acoustic characteristics of acoustic output of the subject, and processing these measured characteristics to determine the emotional state of the subject.

In another embodiment of the subject invention, a method of determining a physiological state of a subject is provided. In an embodiment, the method includes measuring one or more one or more physiological characteristics of the subject and/or measuring one or more acoustic characteristics of acoustic output of the subject, and processing these measured characteristics to determine the physiological state of the subject. In a particular embodiment, the method includes: measuring one or more physiological characteristics of the subject; creating a corresponding one or more predicted physiological characteristics of the subject based on the measured one or more physiological characteristics of the subject; measuring one or more acoustic characteristics of acoustic output of the subject; refining the corresponding one or more predicted physiological characteristics based on the measured one or more acoustic characteristics; and determining the physiological state of the subject based on the refined one or more physiological characteristics of the subject.

In another embodiment of the subject invention, a method of determining physiological characteristics of a subject is provided. In an embodiment, the method includes: measuring one or more physiological characteristics of the subject; creating a corresponding one or more predicted physiological characteristics of the subject based on the measured one or more physiological characteristics of the subject; measuring one or more acoustic characteristics of acoustic output of the subject; and normalizing the corresponding one or more predicted physiological characteristics based on the measured one or more acoustic characteristics.

In embodiments, the physiological measurements can be taken via a physiological data acquisition unit, such as the physiological data acquisition unit described above in relation to FIG. 1. The acoustic measurements can be taken via an acoustic data acquisition unit, such as the acoustic data acquisition unit described above in relation to FIG. 1. The measurements can be processed via an information processing unit, such as the information processing unit described above in relation to FIG. 1.

The measured characteristics can be processed in various ways. For example, in an embodiment, one or more of the measured characteristics are first processed to determine a predicted emotional and/or physiological state. Then, one or more additional characteristics are processed to refine the predicted emotional and/or physiological state. For example, the acoustic characteristics can be processed first to determine a predicted emotional state and later the physiological characteristics can be used to refine the predicted emotional state. In an alternative embodiment, the physiological characteristics are processed first to determine a predicted emotional state; and the acoustic characteristics are later used to refine the predicted emotional state. For example, an elevated heart beat can predict an emotional state including excitement and later acoustic information can be used to further describe the predicted emotional state as expressing either fear or surprise.

In another embodiment, one or more acoustic characteristics are processed to determine at least one baseline physiological characteristic for the subject. For example, the acoustic information can be used to determine the gender and/or race of the subject. Then, an appropriate threshold for analyzing the subject's physiological characteristics can be selected based on the gender and/or race of the subject. In yet another embodiment, one or more physiological characteristics are processed to determine at least one baseline acoustic characteristic for acoustic output of the subject. For example, a respiration rate of the subject can be used to determine a baseline speaking rate for the subject.

The measured characteristics can be processed in other ways. For example, a first one or more of the measured characteristics can be normalized or correlated based on a second one or more of the measured characteristics. In a particular embodiment, one or more physiological characteristics are normalized and/or correlated based on at least one acoustic characteristic. In another embodiment, one or more acoustic characteristics are normalized and/or correlated based on at least one physiological characteristic.

According to embodiments of the subject invention, measured characteristics and/or predicted or determined states are associated with particular periods of time. For example, acoustic and/or physiological characteristics can be measured after a particular stimulus, such as a question, is provided to the subject. Then these measurements can be processed in order to determine and/or predict an emotional and/or physiological state of the subject during the particular period of time. Thus, the subject's reaction to a stimulus can be gauged. In embodiments, the measured time period, in which measurements are captured, does not necessarily align with the stimulus time period, in which the stimulus occurs, or the predicted time period, for which a state is determined. For example, a delay can be used to provide time for the subject to react to the stimulus and/or for the reaction to affect the physiological and/or acoustic characteristics exhibited by the subject. Various delay lengths can be used for various applications. In a particular embodiment, a delay of about two seconds is used between when the stimulus occurs and measurement begins. In a particular embodiment, measurements commence within three seconds of the beginning or ending of the stimulus time period. In another embodiment, measurements begin as soon as the stimulus time period expires, i.e., the stimulus is complete. In another embodiment, measurements are taken for a greater period of time—including, potentially, times before, during, and after the stimulus time period—and later the measurements are associated with the timing of the stimulus. For example, physiological measurements can be taken before the beginning of the stimulus time period to provide a baseline. Later, additional measurements can be taken. If a change is noted, the timing of the change can be considered and associated with the timing of the stimulus. In an embodiment, the system notes the change and directs a physiological data acquisition unit to take additional or more frequent measurements for a period of time. Acoustic measurements can be triggered when speech by the subject first occurs following the beginning or completion of the stimulus time period.

Various measured time period durations can be used for various applications. The length of the needed time period and/or delay can vary based on the type of measurement to be taken. In a particular embodiment, the measured time period lasts 10 to 20 seconds. In another it lasts, 3 to 4 seconds. In yet another it lasts, about 5 seconds. In an embodiment, a plurality of measurements are taken during the measured time period. In this case, each measurement can correspond to a sub-measured time period within the measured time period. For example heartbeat can be measured for the first five seconds of the measured time period, while respiration rate can be measured for the first ten seconds of the measured time period. Some characteristics can be measured several times during the measured time period while others can be measured just once. For example, in a particular embodiment, one or more acoustic characteristics are measured twice during a 20 second measured time period, each measurement occurring over a 3 to 4 second sub-measured time period. Concurrently, one or more physiological characteristics are measured over a 10 to 20 second sub-measured time period within the 20 second measured time period. The plurality of measurements can then be processed as discussed above in order to determine an emotional and/or physiological state of the subject and/or the subject's reaction to a stimulus.

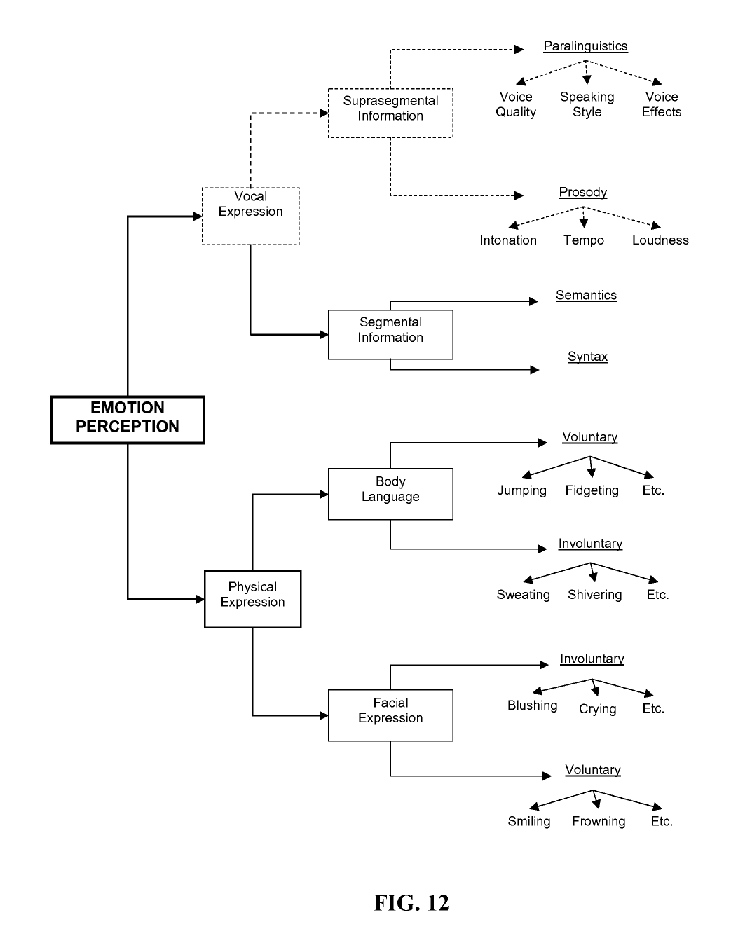

A subject's emotional and/or physiological state can be perceived in various ways, as shown in FIG. 12. Various characteristics can be measured to determine a subjects emotional or physiological state. Such measured characteristics can include physiological characteristics, such as heartbeat, respiration, temperature, and galvanic skin response. Such measured characteristics can also include acoustic characteristics of acoustic output of the subject. In an embodiment, the acoustic output of the subject includes speech of the subject and acoustic characteristics of the speech of the subject are measured. In a further embodiment, suprasegmental properties of the speech of the subject are measured, such as the acoustic cues discussed in Table 1. In an embodiment, such measured characteristics are measured in a non-contact manner. In an embodiment, the acoustic measurements and/or physiological measurements are processed in real time.

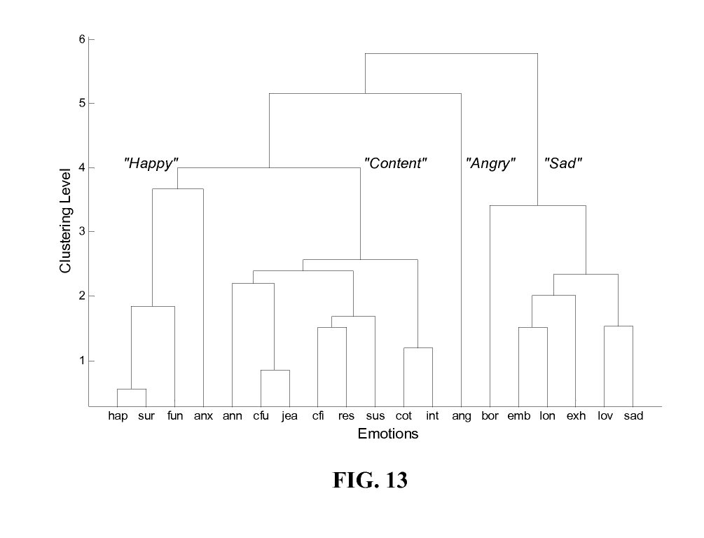

Emotions can be categorized in various ways, for example as taught in International Application No. PCT/US2010/038893, which is incorporated by reference herein in its entirety, including any figures, tables, or drawings. An example is shown in FIG. 13, in which ang=angry; ann=annoyed; anx=anxious; bor=bored; cfi=confident; cfu=confused; cot=content; emb=embarrassed; exh=exhausted; fun=funny; hap=happy; int=interested; jea=jealous; lon=lonely; lov=love; res=respectful; sad=sad; sur=surprised; and sus=suspicious are categorized into categories and sub-categories according to the results of an investigation. Other categorizations or emotional definitions can be used.

An acoustic space having one or more dimensions, where each dimension of the one or more dimensions of the acoustic space corresponds to at least one baseline acoustic characteristic can be created and provided for providing baseline acoustic characteristics, for example as taught in International Application No. PCT/US2010/038893, which is incorporated by reference herein in its entirety, including any figures, tables, or drawings. The acoustic space can be created, or modified, by analyzing training data to determine, or modify, repetitively, the at least one baseline acoustic characteristic for each of the one or more dimensions of the acoustic space.

The emotion state of speaker can include emotions, categories of emotions, and/or intensities of emotions. In a particular embodiment, the emotion state of the speaker includes at least one magnitude along a corresponding at least one of the one or more dimensions within the acoustic space. The baseline acoustic characteristic for each dimension of the one or more dimensions can affect perception of the emotion state. The training data can incorporate one or more training utterances of speech. The training utterance of speech can be spoken by the speaker, or by persons other than the speaker. The utterance of speech from the speaker can include one or more of utterances of speech. For example, a segment of speech from the subject utterance of speech can be selected as a training utterance.

The acoustic characteristic of the subject utterance of speech can include a suprasegmental property of the subject utterance of speech, and a corresponding baseline acoustic characteristic can include a corresponding suprasegmental property. The acoustic characteristic of the subject utterance of speech can be one or more of the following: fundamental frequency, pitch, intensity, loudness, speaking rate, number of peaks in the pitch, intensity contour, loudness contour, pitch contour, fundamental frequency contour, attack of the intensity contour, attack of the loudness contour, attack of the pitch contour, attack of the fundamental frequency contour, fall the intensity contour, fall of the loudness contour, fall of the pitch contour, fall of the fundamental frequency contour, duty cycle of the peaks in the pitch, normalized minimum pitch, normalized maximum of pitch, cepstral peak prominence (CPP), and spectral slope.

One method of obtaining the baseline acoustic measures is via a database of third party speakers (also referred to as a “training” set), for example as taught in International Application No. PCT/US2010/038893, which is incorporated by reference herein in its entirety, including any figures, tables, or drawings. The speech samples of this database can be used as a comparison group for predicting or classifying the emotion of any new speech sample. For example, the training set can be used to train a machine-learning algorithm. These algorithms may then be used for classification of novel stimuli. Alternatively, the training set may be used to derive classification parameters such as using a linear or non-linear regression. These regression functions may then be used to classify novel stimuli.

A second method of computing a baseline is by using a small segment (or an average of values across a few small segments) of the target speaker as the baseline, for example as taught in International Application No. PCT/US2010/038893, which is incorporated by reference herein in its entirety, including any figures, tables, or drawings. All samples are then compared to this baseline. This can allow monitoring of how emotion may change across a conversation (relative to the baseline).

The number of emotion categories can depend varying on the information used for decision-making. Using suprasegmental information alone can lead to categorization of, for example, up to six emotion categories (happy, content, sad, angry, anxious, and bored). Inclusion of segmental information (words/phonemes or other semantic information) or non-verbal information (e.g. laughter) can provides new information that may be used to further refine the number of categories. The emotions that can be classified when word/speech and laughter recognition is used can include disgust, surprise, funny, love, panic fear, and confused.

For a given speech input, two kinds of information may be determined: (1) The “category” or type of emotion and, (2) the “magnitude” or amount of emotion present.

Table 1 includes parameters that may be used to derive each emotion and/or emotion magnitude. Importantly, parameters such as alpha ratio, speaking rate, minimum pitch, and attack time are used in direct form or after normalization. Please note that this list is not exclusive and only reflects the variables that were found to have the greatest contribution to emotion detection in our study.

Emotion categorization and estimates of emotion magnitude may be derived using several techniques (or combinations of various techniques). These include, but are not limited to, (1) Linear and non-linear regressions, (2) Discriminant analyses and (3) a variety of Machine learning algorithms such as HMM, Support Vector Machines, Artificial Neural Networks, etc., for example as taught in International Application No. PCT/US2010/038893, which is incorporated by reference herein in its entirety, including any figures, tables, or drawings.

Embodiments of the subject invention can allow better understanding of disease and/or other conditions shared by a plurality of subjects. Physiological and/or acoustic measurements (“training data”) can be acquired from a plurality of subjects having a particular condition. These measurements can then be processed using 1) Linear and non-linear regressions, (2) Discriminant analyses and/or (3) a variety of Machine learning algorithms such as HMM, Support Vector Machines, Artificial Neural Networks, etc., to develop a profile for the particular condition. After the profile has been trained in this manner, the profile can then be applied as a diagnostic and/or screening tool for assessing one or more other subjects. In an embodiment, similar measurements (“subject data”) are taken from the other subjects. These measurements can then be applied to the profile in order to predict whether the other subjects also have the particular condition.

In an embodiment, the training and/or subject data can be acquired remotely. For example, in an embodiment, physiological and/or acoustic measurements are acquired via a cell phone, PDA, or other client device. The measurements can then be processed on the device and/or uploaded to a server for further processing. Such methods can allow efficient acquisition of training data. For example, as long as a participant's cell phone, PDA, or other client device is capable of taking the needed measurements, recruiting study participants can be done concurrently with acquiring participant data. A simple phone call to or from an enabled cell phone allows data acquisition. Such methods can also allow efficient acquisition of subject data and/or delivery of subject results. For example, a participant can contact a hotline from an enabled cell phone or other client device. Measurements can be acquired via the client device, for example in response to particular voice prompts. In a further embodiment, the subject data is processed in real time via the client device and/or a remote server and a diagnosis or screening decision is delivered during the same phone call. Where additional follow-up is indicated, such as further testing or a doctor's appointment, such follow-up could be arranged during the same call as well. Such methods could be used to profile, diagnosis, and/or screen for post-traumatic stress disorder and/or other medical and nonmedical conditions.

In an embodiment, one or more of steps of a method of determining an emotional and/or physiological state of a subject are preformed by one or more suitably programmed computers. In a particular embodiment, at least one of the processing, refining, predicting, and/or determining steps is preformed by the one or more suitably programmed computers. Computer-executable instructions for performing these steps can be embodied on one or more computer-readable media as described below. In an embodiment, the one or more suitably programmed computers incorporate a processing system as described below. In an embodiment, the processing system is part of a physiological data acquisition unit, acoustic data acquisition unit, and/or an information processing unit.

Aspects of the invention can be described in the general context of computer-executable instructions, such as program modules, being executed by a computer. Generally, program modules include routines, programs, objects, components, data structures, etc., that perform particular tasks or implement particular abstract data types. Such program modules can be implemented with hardware components, software components, or a combination thereof. Moreover, those skilled in the art will appreciate that the invention can be practiced with a variety of computer-system configurations, including multiprocessor systems, microprocessor-based or programmable-consumer electronics, minicomputers, mainframe computers, and the like. Any number of computer-systems and computer networks are acceptable for use with the present invention.

Specific hardware devices, programming languages, components, processes, protocols, formats, and numerous other details including operating environments and the like are set forth to provide a thorough understanding of the present invention. In other instances, structures, devices, and processes are shown in block-diagram form, rather than in detail, to avoid obscuring the present invention. But an ordinary-skilled artisan would understand that the present invention can be practiced without these specific details. Computer systems, servers, work stations, and other machines can be connected to one another across a communication medium including, for example, a network or networks.

As one skilled in the art will appreciate, embodiments of the present invention can be embodied as, among other things: a method, system, or computer-program product. Accordingly, the embodiments can take the form of a hardware embodiment, a software embodiment, or an embodiment combining software and hardware. In an embodiment, the present invention takes the form of a computer-program product that includes computer-useable instructions embodied on one or more computer-readable media. Methods, data structures, interfaces, and other aspects of the invention described above can be embodied in such a computer-program product.

Computer-readable media include both volatile and nonvolatile media, removable and nonremovable media, and contemplate media readable by a database, a switch, and various other network devices. By way of example, and not limitation, computer-readable media incorporate media implemented in any method or technology for storing information. Examples of stored information include computer-useable instructions, data structures, program modules, and other data representations. Media examples include, but are not limited to, information-delivery media, RAM, ROM, EEPROM, flash memory or other memory technology, CD-ROM, digital versatile discs (DVD), holographic media or other optical disc storage, magnetic cassettes, magnetic tape, magnetic disk storage, and other magnetic storage devices. These technologies can store data momentarily, temporarily, or permanently. In an embodiment, non-transitory media are used.

The invention can be practiced in distributed-computing environments where tasks are performed by remote-processing devices that are linked through a communications network or other communication medium. In a distributed-computing environment, program modules can be located in both local and remote computer-storage media including memory storage devices. The computer-useable instructions form an interface to allow a computer to react according to a source of input. The instructions cooperate with other code segments or modules to initiate a variety of tasks in response to data received in conjunction with the source of the received data.

The present invention can be practiced in a network environment such as a communications network. Such networks are widely used to connect various types of network elements, such as routers, servers, gateways, and so forth. Further, the invention can be practiced in a multi-network environment having various, connected public and/or private networks.

Communication between network elements can be wireless or wireline (wired). As will be appreciated by those skilled in the art, communication networks can take several different forms and can use several different communication protocols.

Embodiments of the subject invention can be embodied in a processing system. Components of the processing system can be housed on a single computer or distributed across a network as is known in the art. In an embodiment, components of the processing system are distributed on computer-readable media. In an embodiment, a user can access the processing system via a client device. In an embodiment, some of the functions or the processing system can be stored and/or executed on such a device. Such devices can take any of a variety of forms. By way of example, a client device may be a desktop, laptop, or tablet computer, a personal digital assistant (PDA), an MP3 player, a communication device such as a telephone, pager, email reader, or text messaging device, or any combination of these or other devices. In an embodiment, a client device can connect to the processing system via a network. As discussed above, the client device may communicate with the network using various access technologies, both wireless and wireline. Moreover, the client device may include one or more input and output interfaces that support user access to the processing system. Such user interfaces can further include various input and output devices which facilitate entry of information by the user or presentation of information to the user. Such input and output devices can include, but are not limited to, a mouse, touch-pad, touch-screen, or other pointing device, a keyboard, a camera, a monitor, a microphone, a speaker, a printer, a scanner, among other such devices. As further discussed above, the client devices can support various styles and types of client applications.

All patents, patent applications, provisional applications, and publications referred to or cited herein are incorporated by reference in their entirety, including any figures, tables, or drawings, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

What is claimed is:

1. A method of determining an emotional state of a subject, comprising:
measuring one or more physiological characteristics of a subject;
measuring one or more acoustic characteristics of acoustic output of the subject; and
processing the measured one or more physiological characteristics and the measured one or more acoustic characteristics to determine an emotional state of the subject.

2. The method according to claim 1, wherein processing the measured one or more physiological characteristics and the measured one or more acoustic characteristics to determine an emotional state of the subject comprises:
processing the measured one or more acoustic characteristics to determine a predicted emotional state of the subject; and
refining and/or verifying the predicted emotional state of the subject based on the measured one or more physiological characteristics to determine the emotional state of the subject.

3. The method according to claim 2, further comprising:
providing an acoustic space having one or more dimensions, wherein each dimension of the one or more dimensions of the acoustic space corresponds to at least one baseline acoustic characteristic; and
comparing each acoustic characteristic of the measured one or more acoustic characteristics to a corresponding one or more baseline acoustic characteristics,
wherein processing the measured one or more acoustic characteristics to determine a predicted emotional state of the subject, comprises predicting an emotional state of the subject based on the comparison, wherein the emotional state of the subject comprises at least one magnitude along a corresponding at least one of the one or more dimensions within the acoustic space.

4. The method according to claim 1, wherein processing the measured one or more physiological characteristics and the measured one or more acoustic characteristics to determine an emotional state of the subject comprises:
processing the measured one or more physiological characteristics to determine a predicted emotional state of the subject; and
refining and/or verifying the predicted emotional state of the subject based on the measured one or more acoustic characteristics to determine the emotional state of the subject.

5. The method according to claim 4, wherein the measured one or more physiological characteristics comprises a heartbeat of the subject, wherein the heartbeat of the subject is processed to determine that the predicted emotional state of the subject comprises excitement, and the predicted emotional state of the subject is refined based on the measured one or more acoustic characteristics to determine that the emotional state of the subject comprises fear.

6. The method according to claim 1, wherein processing the measured one or more physiological characteristics and the measured one or more acoustic characteristics to determine an emotional state of the subject comprises:
determining at least one baseline physiological characteristic based on the measured one or more acoustic characteristics;
processing the measured one or more physiological characteristics with the determined at least one baseline physiological characteristic; and
determining the emotional state of the subject based on processing the measured one or more physiological characteristics with the determined at least one baseline physiological characteristic.

7. The method according to claim 6, wherein determining the at least one baseline physiological characteristic based on the measured one or more acoustic characteristics comprises:
predicting an age and/or gender of the subject based on the measured one or more acoustic characteristics; and
determining the at least one baseline physiological characteristic based on the predicated age and/or gender of the subject.

8. The method according to claim 1, wherein processing the measured one or more physiological characteristics and the measured one or more acoustic characteristics to determine an emotional state of the subject comprises:
determining at least one baseline acoustic characteristic based on the measured one or more physiological characteristics;
processing the measured one or more acoustic characteristics with the determined at least one baseline acoustic characteristic; and
determining an emotional state of the subject based on processing the measured one or more acoustic characteristics with the determined at least one baseline acoustic characteristic.

9. The method according to claim 8, wherein determining the at least one baseline acoustic characteristic based on the measured one or more physiological characteristics comprises:
measuring a respiration rate of the subject; and
determining the at least one baseline acoustic characteristic based on the measured respiration rate of the subject.

10. The method according to claim 9, wherein the at least one baseline acoustic characteristic comprises a speaking rate.

11. The method according to claim 8, further comprising:
providing an acoustic space having one or more dimensions, wherein each at least one baseline acoustic characteristic corresponds to one or more dimensions in the acoustic space, wherein the determined emotional state of the subject comprises at least one magnitude along a corresponding at least one of the one or more dimensions within the acoustic space.

12. The method according to claim 1, wherein processing the measured one or more physiological characteristics and the measured one or more acoustic characteristics to determine an emotional state of the subject comprises:
selecting at least one measured physiological characteristic of the measured one or more physiological characteristics, wherein the selected at least one measured physiological characteristic corresponds to a corresponding at least one measured physiological segment of time within a particular segment of time;
selecting at least one measured acoustic characteristic of the measured one or more acoustics characteristics, wherein the selected at least one measured acoustic characteristic corresponds to a corresponding at least one measured acoustic segment of time within the particular segment of time; and
processing the selected at least one measured physiological characteristic and the selected at least one measured acoustic characteristic to determine the emotional state of the subject.

13. The method according to claim 12, wherein the emotional state of the subject corresponds to the particular segment of time.

14. The method according to claim 13, wherein the particular segment of time corresponds to a 10 to 20 second segment of time.

15. The method according to claim 13, wherein the particular segment of time occurs after a stimulus is provided to the subject.

16. The method according to claim 13, wherein the particular segment of time begins within 3 seconds of the stimulus.

17. The method according to claim 15, wherein the stimulus is a question posed to the subject.

18. The method according to claim 15, further comprising providing the stimulus to the subject.

19. The method according to claim 13, wherein the corresponding at least one measured acoustic segment of time is one measured acoustic segment of time within the particular segment of time, wherein each of the selected at least one measured acoustic characteristics corresponds to the measured acoustic segment of time.

20. The method according to claim 13, wherein the corresponding at least one measured acoustic segment of time comprises a plurality of measured acoustic segments of time within the particular segment of time, wherein each of the selected at least one measured acoustic characteristics corresponds to one of the plurality of measured acoustic segments of time.

21. The method according to claim 19, wherein the corresponding at least one measured physiological segment of time is one measured physiological segment of time within the particular segment of time, wherein each of the selected at least one measured physiological characteristics corresponds to the measured physiological segment of time.

22. The method according to claim 19, wherein the corresponding at least one measured physiological segment of time comprises a plurality of measured physiological segments of time within the particular segment of time, wherein each of the selected at least one measured physiological characteristics corresponds to one of the plurality of measured physiological segments of time.

23. The method according to claim 20, wherein the corresponding at least one measured physiological segment of time is one measured physiological segment of time within the particular segment of time, wherein each of the selected at least one measured physiological characteristics corresponds to the measured physiological segment of time.

24. The method according to claim 20, wherein the corresponding at least one measured physiological segment of time comprises a plurality of measured physiological segments of time within the particular segment of time, wherein each of the selected at least one measured physiological characteristics corresponds to one of the plurality of measured physiological segments of time.

25. The method according to claim 13, further comprising:
selecting an additional at least one measured physiological characteristic of the measured one or more physiological characteristics, wherein the additional at least one measured physiological characteristic corresponds to a corresponding additional at least one measured physiological segment of time within an additional segment of time;
selecting an additional at least one measured acoustic characteristic of the measured one or more acoustics characteristics, wherein the additional at least one measured acoustic characteristic corresponds to a corresponding additional at least one measured acoustic segment of time within the additional segment of time; and
processing the additional at least one measured physiological characteristic and the additional at least one measured acoustic characteristic to determine an additional emotional state of the subject corresponding to the additional segment of time.

26. The method according to claim 25, wherein the particular segment of time and the additional segment of time overlap.

27. The method according to claim 25, wherein the particular segment of time and the additional segment of time do not overlap.

28. The method according to claim 1, where at least one of the one or more physiological characteristics is selected from the group consisting of:
heartbeat, respiration, temperature, and galvanic skin response.

29. The method according to claim 1, wherein the acoustic output of the subject comprises speech of the subject and at least one of the one or more acoustic characteristics is an acoustic characteristic of the speech of the subject.

30. The method according to claim 29, wherein at least one of the one or more acoustic characteristics comprises a suprasegmental property of the speech of the subject.

31. The method according to claim 29, wherein the at least one of the one or more acoustic characteristics is selected from the group consisting of: fundamental frequency, pitch, intensity, loudness, and speaking rate.

32. The method according to claim 29, wherein the at least one of the one or more acoustic characteristics is selected from the group consisting of: number of peaks in the pitch, intensity contour, loudness contour, pitch contour, fundamental frequency contour, attack of the intensity contour, attack of the loudness contour, attack of the pitch contour, attack of the fundamental frequency contour, fall the intensity contour, fall of the loudness contour, fall of the pitch contour, fall of the fundamental frequency contour, duty cycle of the peaks in the pitch, normalized minimum pitch, normalized maximum of pitch, cepstral peak prominence (CPP), and spectral slope.

33. The method according to claim 1, wherein measuring the one or more physiological characteristics is accomplished in a non-contact manner.

34. The method according to claim 33, wherein measuring the one or more physiological characteristics comprises:
transmitting an RF signal towards the subject;
receiving a reflected RF signal from the subject;
identifying different orders of harmonics caused by a non-linear effect in the reflected RF signal; and
determining an amplitude of a periodic movement of the target from the identified different orders of harmonics.

35. The method according to claim 33, wherein measuring the one or more physiological characteristics comprises:
transmitting a signal towards the subject;
receiving a reflected signal from the subject;
reconstructing a complex signal from an I channel and a Q channel for the received reflected signal;
applying a Fourier transform to the reconstructed signal to obtain the detected spectrum;
extracting angular information of the reconstructed complex signal; and
obtaining original vibration information by analyzing the angular information.

36. The method according to claim 1, wherein measuring the one or more acoustic characteristics is accomplished in a non-contact manner.

37. The method according to claim 1, wherein processing the measured one or more physiological characteristics and the measured one or more acoustic characteristics comprises correlating the measured one or more physiological characteristics with the measured one or more acoustic characteristics.

38. The method according to claim 1, wherein processing the measured one or more physiological characteristics and the measured one or more acoustic characteristics comprises normalizing the measured one or more physiological characteristics based on the measured one or more acoustic characteristics.

39. The method according to claim 1, wherein processing the measured one or more physiological characteristics and the measured one or more acoustic characteristics comprises normalizing the measured one or more acoustic characteristics based on the measured one or more physiological characteristics.

40. A method of determining a physiological state of a subject, comprising:
measuring one or more physiological characteristics of a subject;
creating a corresponding one or more predicted physiological characteristics of the subject based on the measured one or more physiological characteristics of the subject;
measuring one or more acoustic characteristics of acoustic output of the subject;
refining the corresponding one or more predicted physiological characteristics based on the measured one or more acoustic characteristics; and
determining a physiological state of the subject based on the refined one or more physiological characteristics of the subject.

41. The method according to claim 40, wherein refining the one or more physiological characteristics based on the measured one or more acoustic characteristics comprises normalizing the predicted one or more physiological characteristics based on the measured one or more acoustic characteristics.

42. A method of determining physiological characteristics of a subject, comprising:
measuring one or more physiological characteristics of a subject;
creating a corresponding one or more predicted physiological characteristics of the subject based on the measured one or more physiological characteristics of the subject;
measuring one or more acoustic characteristics of acoustic output of the subject; and
normalizing the corresponding one or more predicted physiological characteristics based on the measured one or more acoustic characteristics.

43. A method of determining a subject's emotional state, comprising:
measuring one or more acoustic characteristics of acoustic output of a subject, wherein the measured one or more acoustic characteristics corresponds to a corresponding one or more measured acoustic segments of time within a particular segment of time, wherein the particular segment of time occurs after a stimulus; and
processing the measured one or more acoustic characteristics to determine an emotional state of the subject, wherein the emotional state of the subject corresponds to the particular segment of time.

44. A method of determining a subject's emotional state, comprising:
measuring one or more physiological characteristics a subject, wherein the measured one or more physiological characteristics corresponds to a corresponding one or more measured physiological segments of time within a particular segment of time, wherein the particular segment of time occurs after a stimulus; and
processing the measured one or more physiological characteristics to determine an emotional state of the subject, wherein the emotional state of the subject corresponds to the particular segment of time.

45. An apparatus for determining a subject's emotional state, comprising:
a physiological data acquisition unit, wherein the physiological data acquisition unit acquires physiological data of a subject;
an acoustic data acquisition unit, wherein the acoustic data acquisition unit acquires acoustic data of a subject; and
an information processing unit, wherein the information processing unit receives and processes the physiological data and/or acoustic data, and outputs an indication of the subject's emotional state.

46. The apparatus according to claim 45, wherein the physiological data acquisition unit acquires physiological data in a non-contact manner.

47. The apparatus according to claim 45, wherein the acoustic data acquisition unit acquires acoustic data in a non-contact manner.

48. The apparatus according to claim 45, wherein the physiological data acquisition unit comprises a non-contact physiological data detection radar.

49. The apparatus according to claim 45, wherein the acoustic data acquisition unit comprises a transducer and a storage device.

50. The apparatus according to claim 49, wherein the transducer is a microphone.

51. The apparatus according to claim 45, where at least one of the physiological data acquired is selected from the group consisting of: heartbeat, respiration, temperature, and galvanic skin response.

52. The apparatus according to claim 45, wherein the acoustic data acquired is speech.

53. The apparatus according to claim 45, wherein the apparatus measures physiological data and acoustic data simultaneously.
